(19) United States
(12) Patent Application Publication
(10) Pub. No.: US 2026/0086097 A1
(43) Pub. Date: Mar. 26, 2026

(54) N-TERMINAL MULTIFUNCTIONAL CONJUGATION OF PROTEINS AND PEPTIDES (FOR BIOSENSING)

(71) Applicant: Roswell Biotechnologies, Inc., San Diego, CA (US)

(72) Inventors: Jose Rizo, San Diego, CA (US); Prem Kumar Sinha, San Diego, CA (US); Kathryne Byrne-Bailey, San Diego, CA (US); Cruz Lamberto Sigala, San Diego, CA (US); Carl Fuller, Berkeley Heights, NJ (US); Barry Merriman, San Diego, CA (US)

(73) Assignee: Roswell Biotechnologies, Inc., San Diego, CA (US)

(21) Appl. No.: 19/109,656

(22) PCT Filed: Sep. 8, 2023
(86) PCT No.: PCT/US2023/032293

§ 371 Date: Mar. 7, 2025

Related U.S. Application Data
(60) Provisional application No. 63/404,944, filed on Sep. 8, 2022.

(51) Int. Cl. G01N 33/68; C12Q 1/48; G01N 27/327; G01N 33/543

(52) U.S. Cl. CPC G01N 33/6845; C12Q 1/485; G01N 27/3276; G01N 33/5438; G01N 2333/161; G01N 2333/5412; G01N 2333/9126

(57) ABSTRACT

In various embodiments alpha helical peptide-based bridges for molecular biosensing “on-chip” are disclosed. The Peptide-based bridges serve as common bridges for great diversity of biosensing applications and targets including nucleic acids, proteins, antigens, antibodies, small molecules. The primary sensor element is preferably a “molecular wire” such as an alpha-helical peptide integrated into a current monitoring circuit. The engineered peptide may contain a central conjugation site for attachment of various probe molecules including nucleic acids, proteins, antigens, antibodies. The probe-containing bridge empowers the sensor to detect interactions with specific target molecules.

### REFERENCE TO CROSS-RELATED APPLICATIONS

The instant application claims priority to, and is a National Stage filing of PCT/US23/32293 filed Sep. 8, 2023, entitled “N-Terminal Multifunctional Conjugation of Proteins and Peptides for Biosensing”, which claimed the filing date benefit of, U.S. Provisional Patent Application Ser. No. 63/404,944 by Jose Rizo et al., entitled ‘N-Terminal Multifunctional Conjugation of Proteins and Peptides for Biosensing’ filed on Sep. 8, 2022, and International Patent Application PCT/US23/14291 by Prem Kumar Sinha et al., entitled “Peptide Based Bridges for Molecular Sensors and Methods for use thereof” filed on Mar. 1, 2023, the disclosures of all the foregoing is incorporated herein by reference in their entireties for all purposes.

### FIELD

The present disclosure is generally directed to molecular sensors in which a probe is bound to an amino acid bridge molecule in a molecular circuit and binding of a target or ligand to the probe is detectible by monitoring at least one parameter of the molecular circuit.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

The broad field of molecular electronics was introduced in the 1970's by Aviram and Ratner. Molecular electronics achieves the ultimate scaling down of electrical circuits by using single molecules as circuit components. Molecular circuits comprising single molecule components can function diversely as switches, rectifiers, actuators and sensors, depending on the nature of the molecule. Of particular interest is the application of such circuits as biosensors, where molecular interactions provide a basis for single biomolecule sensing.

Biosensing is a rapidly evolving methodology for monitoring biochemical pathways and quantifying biomolecules, particularly those of medical relevance. As such, the need to develop probes that are biologically representative of the systems of interest while remaining synthetically accessible has led to the emergence of the field of bioconjugation as a powerful tool that intersects biology, synthetic chemistry, and biochemistry. In the past, the issue of assessing synthetic viability has relied on the adaptation of well-established synthetic techniques to ligate probes of interest. Probes, including but not limited to peptides, oligos, and other ligands of interest, were integrated into biosensing applications utilizing synthetic techniques that targeted specific functional groups on the target.

These techniques have traditionally employed activated esters to target amine containing residues, Michael-Acceptors to target thiols, and more recently, redox-mediated coupling to target phenolic amino acid residues. While granting a moderate to appreciable degree of chemoselectivity, these synthetic methods rarely provide regioselective ligation without secondary factors being considered. Such considerations include protein engineering to remove or hinder the reactivity of competing residues with respect to the synthetic technique that is being employed, or the modification of these residues via synthetic or enzymatic techniques to modulate reactivity and selectivity. While the non-selective ligation may suffice for certain applications, such as those simply probing for binding events, the non-specific incorporation of substrates, specifically enzymes, may lead to complications in systems in which the activity of the enzyme may be altered by the location of the modification. This is particularly a concern in systems that are sensitive to the variance in dynamics caused by populations of probe-sensor conjugates that are modified at varying positions, such as single-molecule biosensor systems.

To this end, great efforts have been made to develop a method that allows for both chemoselective and regioselective ligation of protein-based probes, such that a homogeneous population of probe-conjugate species is afforded. Two key factors were heavily weighted. First, the method must be minimally interfering in terms of the chemical modification on the protein substrate, and second, the method should not require the protein to be engineered to attach any handles (chemically reactive moieties). With these constraints in mind, the primary amine present on the N-Terminus was identified as a suitable target for bioconjugation.

While the literature is rich in previous methodologies having similar aim, most rely on synthetic strategies that either incorporate moieties that are prone to cross reactivity, thus yielding poor to moderate yield and selectivity, or moieties that have poor water solubility, thus requiring the use of organic solvents and limiting their utility with regards to applications employing larger hydrophilic biomolecules. Likewise, as similar concern is the synthetic accessibility of the proposed reagents in question. Although some of these synthetic strategies can be employed to install simple modifications on biomolecules of interest, the practical feasibility for installing multifunctional molecules is greatly limited by the relative complexity of synthesizing these linker reagents. However, some of these strategies are prime choices for the application of chemo- and regio-selective probe ligation for biosensing applications. For example, both Mikkelson et al. and Ohana et al. have reported two distinct moieties that afford N-terminus selective peptide modification. Importantly, however, the focus in these reports was on demonstrating the modification of small peptides with simple functionalities.

Much more work and information are needed regarding larger polypeptides, in particular functional proteins, and in particular, how the modifications might be implemented in useful applications. The inventions described herein address this need. Some embodiments herein are directed to the implementation of these selected moieties in rationally designed multifunctional linker molecules for use in a broader ligation regime, particularly for the use in biosensing applications. As described in detail herein below, further novel embodiments of the invention are described herein.

### SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

The present disclosure generally relates to biosensors, systems including the biosensors, and to methods of using the biosensors and systems. In various embodiments, binding probe-based circuits are disclosed. Exemplary biosensors can be used to, for example, detect the binding of a molecule of interest (herein encompassed by the term ‘target) with the probe or a binding partner or ligand of the probe of the sensor.

One particular aspect of the invention includes the use of modified phenolic esters to selectively modify polypeptides at the n-terminal residue. In another aspect, conjugates of a molecular wire and binding probe molecule are described herein, as well as methods of making these conjugates. In another aspect, methods of making a sensor device (e.g. biosensor) that can incorporate and use these modified polypeptides are provided. In another aspect, biosensors are provided that incorporate such modified polypeptides.

In some embodiments, a biosensor device includes i) a current carrying molecular structure comprising a metal contact on the surface of an electrode, wherein the metal contact is coupled to a molecular wire that comprises a conjugation site, ii) a binding probe molecule connected to the molecular wire, where a circuit is formed that capable of detecting and/or obtaining detailed information about the binding of the binding probe molecule to a target ligand or binding partner.

In some embodiments, the current carrying structure includes a positive electrode and a negative electrode. The positive electrode may have a metal contact on its surface, and the negative electrode may also have a metal contact on its surface.

In some embodiments of the biosensor device the molecular wire is an alpha helical peptide.

In some embodiments of the biosensor device the molecular wire is polynucleotide such as single or double stranded DNA, RNA, origami, or polynucleotides with modified backbones.

In some embodiments of the biosensor device the molecular wire is a non-peptide bridge molecule such as graphene, indium oxide ribbons, carbon nanotube, DNA/Polynucleotide.

In some embodiments of the biosensor device the alpha helical peptide comprises one or more conjugation site for coupling to a polyfunctional linker molecule.

In some embodiments of the biosensor device, the conjugation of the binding probe molecule to the molecular wire is through a polyfunctional linker molecule covalently linked at one end to the N-terminus of the binding probe molecule and at the other end to a conjugation site in the molecular wire.

In further embodiments, the polyfunctional linker molecule comprises one or more chemically reactive moiety, handles or surface modifying/binding motifs. In some exemplary embodiments, the polyfunctional linker molecule includes a vinyl boronate linker. In other exemplary embodiments, the polyfunctional linker molecule includes an activated phenol linker. In certain embodiments, one or more reactive moiety has a mixed anhydride such as a sulfonamide or sulfonic acid that is meta to the phenolic oxygen, and proximal to one or more electron withdrawing groups, including but not limited to halogens, polyfluorinated hydrocarbons, nitriles, carboxylic acids and derivatives, and nitro groups. In certain embodiments, the polyfunctional linker molecule has a reactive moiety with a E-styrenyl with a para-electron donating moiety, or an E-vinyl boronate derived from an α-nucleophile/electrophile alkyne that is reacted with a linker that contains one or more reactive groups that can undergo selective ‘click’ style chemistries and be utilized as a scaffold for further expansion.

In some embodiments, the binding probe molecule comprises a protein, peptide, polypeptide, and protein complexes of biological or synthetic origin. In selected embodiments, the binding probe molecule is a polypeptide or protein, including but not limited to a polymerase, a viral antigen, an antibody. Examples of binding probe molecules useful herein include DNA polymerases, the HIV-1 p24 viral antigen, an anti-IL-6 antibody or binding fragment thereof, or protein A.

In other embodiments, molecular wire and binding probe molecule conjugates for use in biosensors are described herein. In an exemplary embodiment, the molecular wire comprises an alpha helical peptide having a conjugation site and the binding probe molecule is a protein having a N-terminus, and the conjugation of the binding probe molecule to the alpha helical peptide is through a polyfunctional linker molecule covalently linked at one end to the N-terminus of the binding probe molecule and at the other end to a conjugation site in the alpha helical peptide. Suitable binding probe molecules to be used in these conjugates include, but are not limited to enzymes, antibodies or binding portions thereof, selected antigens, protein A. Examples of binding probe molecules used in the above embodiment further include a DNA polymerase, the HIV-1 p24 viral antigen, or an anti-IL-6 antibody or binding fragment thereof.

In another aspect, methods of making molecular wire and binding probe conjugates as described above are provided. An example of making such a conjugate includes the steps of synthesis of a polyfunctional linker molecule comprising one or more chemically reactive moiety/handle; selecting a binding probe molecule of interest; reacting the binding probe molecule with bifunctional linker reagent under selected conditions to produce an clickable intermediate binding probe molecule suitable for carbon-hetero bond formation reactions or click chemistry; reacting the clickable intermediate binding probe molecule with a pre-selected molecular wire under appropriate reaction conditions, and purifying the molecular wire and binding probe conjugate. These conjugates can then be used to make specific biosensors by reacting a sensor device with the molecular wire and binding probe conjugate to produce a biosensor for detecting a binding target molecule or ligand that binds to the binding probe molecule.

In yet another embodiment, a method of detection of a target is provided that includes selecting a suitable biosensor such as one described herein, initiating at least one of a voltage or a current through the biosensor, exposing the biosensor to a sample suspected of containing a target of interest, the application of voltage on the sensor, and measuring an electrical change in the circuit.

### BRIEF DESCRIPTION OF THE DRAWINGS

FIG. 1 is a brief scheme of the generalized strategy for synthesizing the primary multifunctional linkers. The synthesis shown utilizes N-capping strategies to enable a diverse set of bioconjugations. The first step (A) illustrates the synthesis of an activated phenol yielding a linker with a reactive handle. The second step (B) shows the synthesis of a bifunctional vinyl boronate linker yielding a further refined linker with a reactive handle. The dotted portion denotes the reactive handle and the area boxed in solid lines denotes the generalizable scaffold for further bioconjugation reactions.

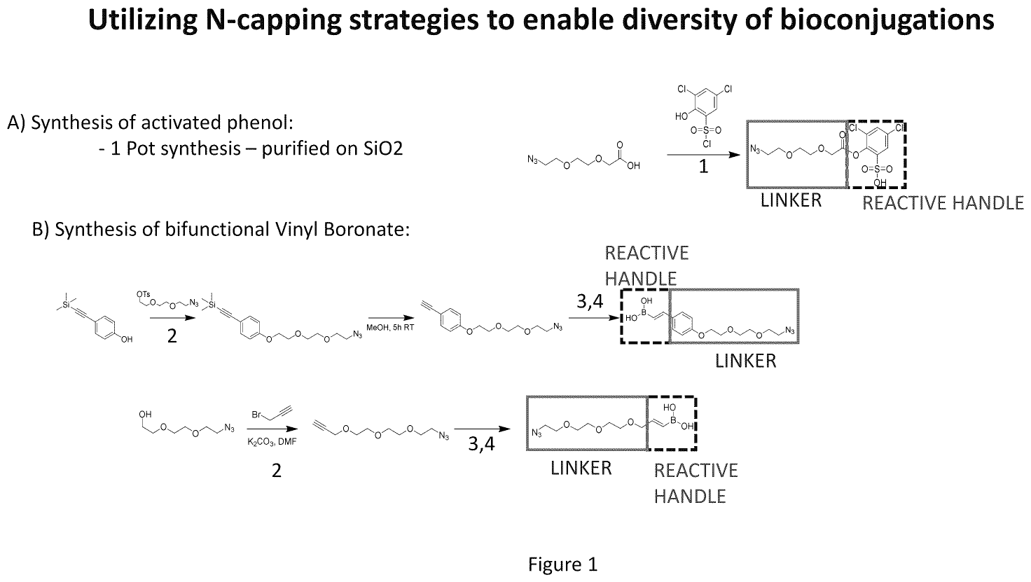

FIG. 2 is a generalized bioconjugation strategy for regio- and chemio-selectively ligating a protein of interest and conjugating it onto a surface binding substrate, such as but not limited to, a molecular wire for biosensing purposes. The first step illustrates the synthesis via an activated phenol and the second step shows the synthesis via an activated vinyl boronate.

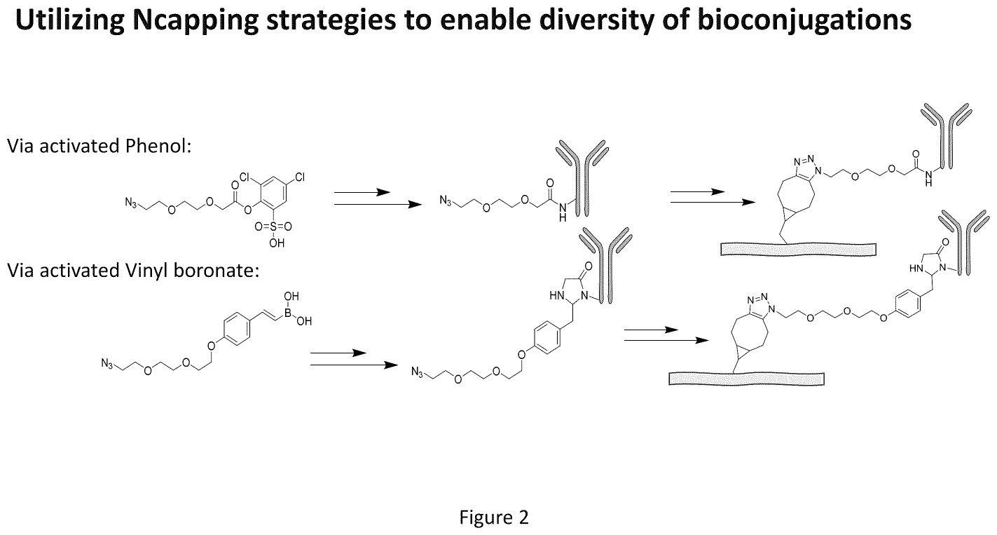

FIG. 3 depicts surface modification/or conjugation anchors for biosensing on various electrode surfaces.

FIG. 4 depicts two representative strategies for on-chip attachment of N-terminally ligated probe.

FIG. 5 is an example showing the representative selective conjugation of an enzyme, in this case, the Bst polymerase with a modified short linker A, and the molecular wire for biosensing purposes. This includes the quality and purification data including FPLC chromatogram shown on the left, and SDS-PAGE gel shown on the right. In the SDS-PAGE gel, lane 1 is a mw ladder/marker, lane 2 is the starting bridge material, lane 3 is the starting Bst polymerase, lane 4 is the reaction mixture, and lanes 5-9 represent different FPLC fractions.

FIG. 6 shows the quality and purification data for the conjugation product of an enzyme, in this case, a polymerase with a modified longer linker B and molecular wire. (FPLC chromatogram on left and SDS-PAGE gel on right). In the SDS-PAGE gel, lane 1 is a mw protein ladder, lanes 2-4 is the starting bridge material, and lanes 5-8 represent different FPLC fraction C9.

FIG. 7 shows result of polymerase activity assay highlighting relative rate of incorporation against dATP. The enzymes used in this case are starting Bst Polymerase (left) and the two N-terminally conjugated Bst polymerase to the bridge peptide via a short linker A (conjugate A, middle) and a longer linker (conjugate B, right).

FIG. 8 shows isolation of a bridge conjugated with HIV-1 p24 antigen. In the SDS-PAGE gel, lane 1 is HIV p24; lane 2 is a protein ladder/marker, and lanes 3-10 represent FPLC fractions C9-D3.

FIG. 9 is a schematic showing HIV-1 p24 antigen attached to the bridge peptide. The left panel is a schematic showing a bridge-probe construct for HIV-1 p24 antibody target. The binding response curve from various chips are also shown by plotting the fraction of the time bound versus the antibody concentration (right panel).

FIG. 10 shows isolation of a bridge peptide conjugated to the N-terminus of an anti-IL-6 antibody.

FIG. 11 is a schematic showing anti-IL-6 antibody attached to the bridge peptide (left panel). The binding response curve from a typical sensor along with a dose-response curve are shown by plotting the fraction of the time bound versus the IL-6 antigen concentration (right panel).

FIG. 12 is a schematic showing Protein A attached to the bridge peptide (left panel). The binding response curve from a typical sensor along with a dose-response curve are shown by plotting the fraction of the time bound versus the IgG antibody concentration (right panel).

FIG. 13 is a schematic that illustrates the general concept of engaging a binding probe molecule onto a molecular wire in an electronic circuit, to act as a sensor capable of detecting a binding event between the probe binding and its target, in accordance with various embodiments. As shown, a polyfunctional linker molecule is covalently linked at one end to the N-terminus of the binding probe molecule and at the other end to a conjugation site in the molecular wire spanning the positive and negative electrodes.

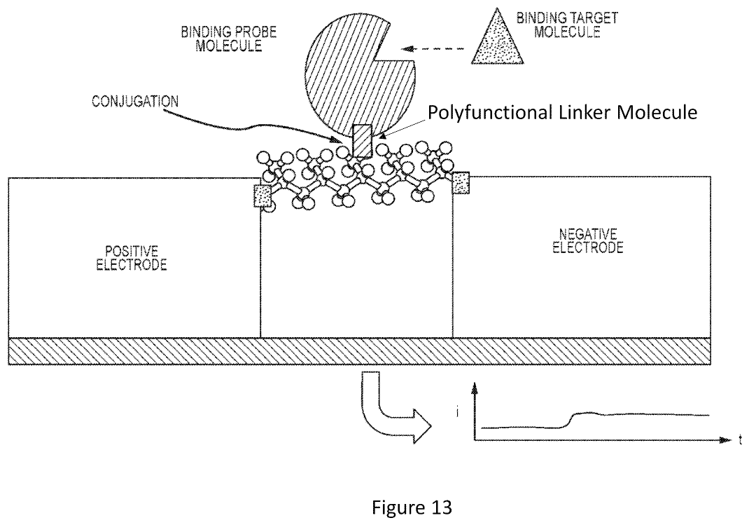

### DETAILED DESCRIPTION

Various aspects of the invention will now be described with reference to the following section which will be understood to be provided by way of illustration only and not to constitute a limitation on the scope of the invention.

As used herein, the term “bridge molecule” or ‘molecular wire’ or ‘nanowire’ can be used interchangeable herein and refers to a molecular wire or other electrically conducting molecule that may be used to make a conducting connection. Numerous molecular wires are described in detail herein. Molecules that function as molecular wires include, but are not limited to, peptide alpha helices, long peptides or polypeptides, modified variants of the proceeding, graphene nanoribbons, pilin filaments or bacterial nanowires, nucleic acids (natural or modified), double stranded DNA, other multichain proteins or conjugates of multiple single-chain proteins, antibodies, carbon nanotubes e.g., single-walled carbon nanotubes (CNTs, SWCNTs), semiconductor layers such as transition metal dichalcogenides (TMD) or other semiconductor nanoribbons or nanowires, or conducting polymers such as polythiophene, poly(3,4-ethylenedioxythiophene (PEDOT) or other synthetic electrically conducting polymers. Such molecules may include attachment groups, i.e., functionality that provide for specific attachment to, and/or self-assembly to, nanoelectrodes or contacts (e.g. metal) such as islands or deposits thereon. Various embodiments described in greater detail herein are directed to the particular conjugation of the binding probe molecule to the bridge molecule or molecular wire.

As used herein, it should be understood that ‘clickable’ or click-chemistry compatible intermediates, structures, functional groups, monomers, oligomers, etc., refer to compounds, materials, etc. that are structurally characterized by including one or more chemical moieties suitable for participation in a click-chemistry reaction. In embodiments where copper-catalyzed azide-alkyne cycloaddition (CuAAC) is the click-chemistry employed for functionalizing materials as disclosed herein, the “clickable” compounds may include a terminal alkyne and/or terminal azide functional group. Click chemistry′ is further described in U.S. Pat. No. 7,375,234, incorporated by reference in its entirety herein.

An exemplary click-chemistry reaction described herein is CuAAC, although skilled artisans will appreciate that other click-chemistry compatible reactions that would be appreciated as equivalent to CuAAC upon reading these descriptions may be employed without departing from the scope of the inventive concepts described herein. For instance, in various embodiments click-chemistry compatible reactions may include CuAAC, strain-promoted azide-alkyne cycloaddition (SPAAC), strain-promoted alkyne-nitrone cycloaddition (SPANC), strained alkene reactions such as alkene-azide cycloaddition, etc. Click-chemistry compatible reactions may also be considered to include alkene-tetrazine inverse-demand Diers-Alder reactions, alkene-tetrazole photoclick reactions, Michael additions of thiols, nucleophilic substitution of thiols with amines, and certain Diels-Alder reactions, etc. such as disclosed by Becer, et al. “Click chemistry beyond metal-catalyzed cycloaddition.” Angew. Chem. Int. Ed. 2009, 48: p. 4900-4908, and equivalents thereof as would be understood by a person having ordinary skill in the art upon reading the present disclosures. Accordingly, click-chemistry compatible groups, compounds, etc. should be understood to include one or more suitable chemical moieties conveying capability to participate in any combination of the foregoing exemplary click chemistries, in various embodiments.

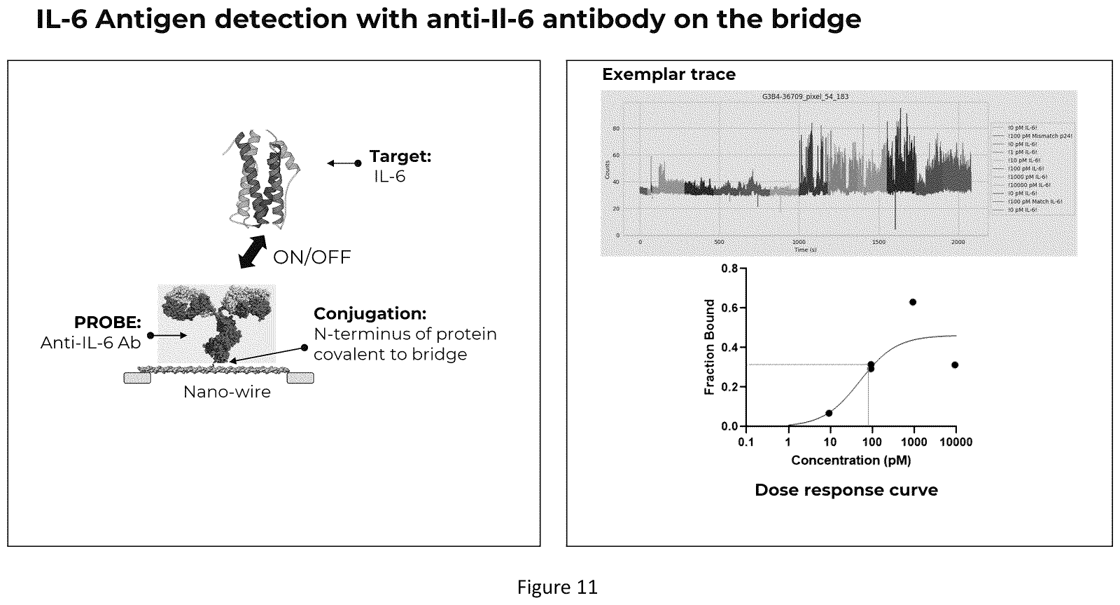

In various embodiments of the present disclosure, a molecular sensor comprises a binding probe molecule connected by a bridge molecule or nanowire to a current carrying structure (e.g. electrodes) to complete a circuit. Interactions of the binding probe molecule with ligand or binding target molecule are detectable as changes in the current or other electrical parameter measured across the circuit (see FIG. 11). The binding probe may be, for example, an enzyme (e.g. polymerase), an antibody, an antigen or any other polypeptide or protein conjugated to a molecular wire or bridge molecule as described. Another exemplary embodiment (not shown) differs from the general concept of a molecular electronic circuit in that the enzyme is directly conjugated or “wired” to both the positive and negative electrodes rather than bonded to a molecular wire that spans the gap between the electrodes to complete a circuit.

In various aspects of the disclosure, at least one of a voltage or a current is initiated in a probe-based molecular circuit. When a target interacts with the probe, electrical changes in the circuit are sensed. These electrical changes, or informative electrical signals, may include current, voltage, impedance, conductivity, resistance, capacitance, or the like. In some examples, a voltage is initiated in the circuit and then changes in the current through the circuit are measured as substrates interact with the binding probe. In other examples, a current is initiated in the circuit, and changes to voltage in the circuit are measured as substrates interact with the enzyme. In other examples, impedance, conductivity, or resistance is measured. In examples wherein the circuit further comprises a gate electrode, such as positioned underneath the gap between the positive and negative electrodes, at least one of a voltage or current may be applied to the gate electrode, and voltage, current, impedance, conductivity, resistance, or other electrical change in the circuit may be measured as substrates interact with the binding probe. Suitable circuits are described in Applicant's prior related patent applications and patents, including U.S. Pat. Nos. 10,036,064, 10,508,296, 10,648,941, 10,584,410, 10,913,966, 11,143,617, and WO/2020/146823A9, all incorporated by reference herein in their entirety.

Certain methods provided herein are directed to the use of modified phenolic esters to selectively modify polypeptides at the n-terminal residue. As noted previously, Mikkelson et al. have demonstrated the ability of modified phenolic esters to selectively modify short peptides at the n-terminal residue. This selectivity is the culmination of multiple factors that have been taken into consideration. As they report, the 3,5-dichloro-2-hydroxy-benzenesulfonic acid esters (PSEs) address the problems associated with traditional activated esters, such as NHS esters. A fundamental drawback of utilizing traditional activated esters is the poor regio- and chemo-selectivity due in part to high reactivity with competing nucleophiles, including other nucleophilic residues and solvents. PSEs circumvent this in a two-fold manner. This moiety imparts stability through an intramolecular hydrogen bond that is formed between the reagent's carbonyl and the phenol's sulfonyl group. Likewise, the electrophilicity of the reagent is aligned to that of the amine at the n-terminal residue via the addition of electron withdrawing groups, such that the HOMO and LUMO of the respective nucleophile and electrophile favor a reaction at the activated ester's carbonyl when the reaction is carried out at near neutral pH. This is specifically due to the difference in pKa of N-terminal amines and that of the ε-amino groups on lysine, ˜9 and 10.5, respectively. Other factors include improved water solubility imparted by the inclusion of a sulfonyl moiety, as well a relative case of synthesis for multifunctional bioconjugation reagents.

### Bioconjugation Targeting N-Terminus of a Protein of Interest:

There are 3 major steps in the process of bioconjugating N-terminus of a given protein to the bridge:

Step 1. Synthesis of a polyfunctional linker molecule harboring appropriate reactive handles.

Step 2. React and isolate probe of interest with bifunctional linker reagent under selective/non-selective conditions to yield clickable intermediate.

Step 3. React clickable intermediate probe with corresponding sensor molecule, anchor, or binding molecule. Purify to yield biosensor probe conjugate.

Step 1: Synthesis of bioconjugation reagent: The first step is synthesis of an appropriate bioconjugation reagent that harbors suitable reactive handles. Below we are citing examples of workflow towards preparation of activated-phenol linker or vinyl boronic acid-linker. However, the conjugation chemistry is not limited to a specific reagent and can be developed for similar reagent types such as triazolocarbaldehydes, pyridine-carbaldehydes, ethynyl benzaldehydes, oxazolines, and mixed anhydrides.

Activated Phenol: A polyfunctional linker molecule where the main reactive handle is a mixed anhydride containing a Sulfonamide or Sulfonic acid that is meta to the phenolic oxygen, alongside one or more electron withdrawing groups, including but not limited to halogens, polyfluorinated hydrocarbons, nitriles, carboxylic acids and derivatives, and nitro groups. The carbonyl containing portion of the mixed anhydride contains one or more reactive groups that can undergo selective ‘click’ style chemistries and can be utilized as a scaffold for further elaboration.

### Example of Workflow:

Under anhydrous conditions, in an inert atmosphere at OC, the sulfonyl chloride-phenol is added dropwise to a solution of the carboxylic acid-linker of interest and a catalytic amount of a dry tertiary amine in an anhydrous, moderately polar, aliphatic solvent with moderate stirring. Once addition is complete, the reaction is allowed to reach room temperature and stirred until the reaction reaches completion as determined via TLC or HPLC. The reaction is then concentrated in vacuo and the remaining residue is redissolved in a minimal amount of organic solvent. This concentrated solution is then purified utilizing an appropriate chromatographic method to yield the final activated-phenol linker.

Vinyl boronate: a polyfunctional linker molecule where the main reactive handle is comprised of a E-Styrenyl with a para-electron donating moiety, or an E-Vinyl Boronate derived from an α-nucleophile/electrophile alkyne that is reacted with a linker that contains one or more reactive groups that can undergo selective ‘click’ style chemistries and be utilized as a scaffold for further expansion.

### Example of Workflow:

Under anhydrous conditions in an inert atmosphere, Alkyne of interest with a suitable electrophile or nucleophile is added dropwise to a solution of linker with a suitable reaction partner in anhydrous DMF containing a suspension of a large excess of anhydrous potassium carbonate. The solution is heated to 80° C. and stirred vigorously overnight. Once the reaction reaches completion as determined by thin layer chromatographic (TLC) or analytical HPLC analysis, the suspension is filtered, diluted with water, and extracted with ether. The pooled organic extracts are washed thoroughly with brine, then dried over anhydrous sodium sulfate prior to in vacuo concentration. The concentrated crude extract is then purified via an appropriate chromatographic process to yield the intermediate purified Alkyne linker.

The purified alkyne linker is then subjected to catalytic hydroboration by overnight heating in toluene at 50° C. with an excess amount of pinacol borane and a catalytic amount of Carbonylchlorohydrido tris (triphenylphosphine) ruthenium (II) under inert anhydrous conditions. Afterwards, the reaction mixture is allowed to cool and concentrated en vacuo. The remaining residue is redissolved in a minimal amount of solvent. This extract is subsequently washed with a saturated sodium bicarbonate solution, brine solution then dried over anhydrous sodium sulfate. The worked-up extract is then concentrated and purified via an appropriate chromatographic process to yield the intermediate protected vinyl boronate-linker.

The intermediate protected vinyl boronate-linker is then deprotected by dissolving in methanol and cooling to 0° C. followed by the dropwise addition of an excess amount of aqueous potassium hydrogenfluoride. This mixture is allowed to stir for one hour, after which it is diluted with water, flash frozen and subsequently lyophilized. The remaining residue is then extracted with organic solvent and filtered to remove solids. The crude extract is then added to an aqueous suspension of silica gel for couple of hours. The suspension is then filtered, and the filtrate is afterwards extracted with additional organic solvent. The pooled organic extracts are then washed with brine and dried over anhydrous sodium sulfate. The dried organic extract is then concentrated in vacuo to yield the final purified vinyl boronic acid-linker.

### Step 2: Isolation of Clickable Intermediate Containing Probe of Interest:

In the first step, the protein probe of interest is buffer exchanged into a neutral non-amine buffer solution containing any non-nucleophilic detergents and stabilizers as appropriate. Subsequently, a large excess of sodium ascorbate is added as freshly prepared aqueous solution, followed by the addition of excess of linker-vinyl boronate. The resulting mixture is vortexed, centrifuged and allowed to incubate for several hours to overnight at 4° C. or room temperature, as appropriate for the bridge being used. The small molecule reactants are removed via centrifugal diafiltration with an appropriately sized membrane filter and optionally purified via suitable chromatographic steps to obtain capped-clickable probe intermediate.

Alternatively, the capped-clickable probe bridge intermediate can be obtained by buffer exchanging the protein probe of interest into a neutral non-amine buffer solution containing any non-nucleophilic detergents and stabilizers as needed. Afterwards, the activated linker-phenol is added in an anhydrous polar water miscible solvent. The resulting mixture is vortexed, centrifuged and allowed to incubate for several hours to overnight at 4° C. or room temperature, as appropriate for the protein being used. The small molecule reactants are removed via centrifugal diafiltration with an appropriately sized membrane filter and optionally purified via suitable chromatographic steps.

### Step 3: Purification of Biosensor Probe Conjugate:

In the subsequent step, the crude or purified clickable probe intermediate solution is mixed with a solution of the desired conjugation partner containing a compatible reactive functional group, generally but not limited to a strain-promoted cycloaddition compatible alkyne. The probe-partner reaction mixture is then incubated at an appropriate temperature, generally 4° C.-50° C. Once sufficient time has elapsed, the crude reaction mixture is concentrated via diafiltration and purified via Size Exclusion HPLC or FPLC using an isocratic mobile phase containing appropriate buffer such as PBS and stabilizers or detergents as needed. The eluted fractions containing the product of interest are then further concentrated and buffer exchanged into the desired final buffer composition via diafiltration. The purified product solution is quantified via gel densitometry and UV-Vis spectrophotometry and purity is assessed by SDS-PAGE and analytical HPLC.

### Coupling Conjugates to a Molecular Wire:

A conjugate comprising a binding probe molecule and a polyfunctional linker can be attached to a molecular wire described herein including, for example, a non-peptide bridge such as one comprising indium oxide, gold, platinum, ruthenium, graphene, carbon nanotube, nucleic acids, or polynucleotides.

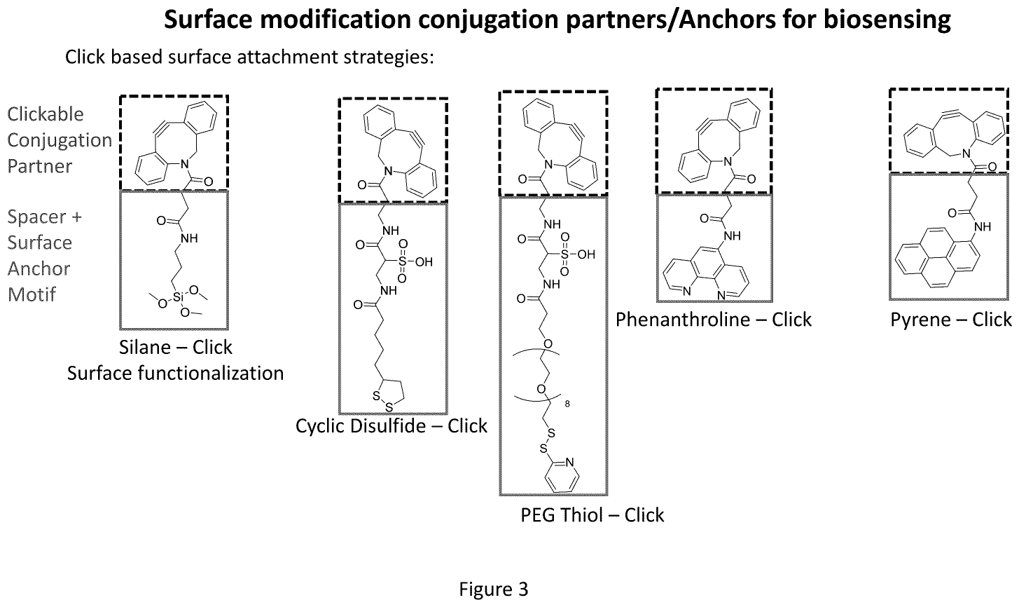

FIG. 3 demonstrates examples of non-peptide including nucleic acid derived conjugation partners that can either be integrated into the polyfunctional linker to act as a surface specific anchor to attach the probe of interest to the electrodes or be integrated as a pre-existing modification of the electrode to enable in-situ ligation of the probe of interest onto the circuit system. These surface anchoring conjugation partners are comprised of a “head” that contains a “click” reactive moiety that is compatible with that which is installed on the probe of interest, and intermediate “spacer” portion that can be linear or branched, which is generally, but not limited to: peptides, nucleic acids, polyether polyols, aliphatic and functionalized alkyl chains, polyesters, polystyrenes, glycans, polyolefins, polyamides, and the like. The “anchor” portion contains one or more moieties that impart surface/material specific binding of the probe of interest through either chemical or physical means. The moieties used depend on the surface or material being utilized in the circuit system. These are generally but not limited to: protected or free thiols or thiol equivalents, such as disulfides or other organosulfur moieties to chemically bind to gold or platinum, polycyclic aromatic hydrocarbons such as pyrene, naphthalene, etc to noncovalently attach to graphene and carbon nanotubes, polycyclic aromatic heterocycles that contain conformationally constrained heteroatoms that can act as polydentate ligands for chemical bonding to ruthenium surfaces, and silane based surface modifications that can be used to install click compatible moieties on a variety of materials.

Additionally, a conjugate comprising a binding probe molecule and a polyfunctional linker can be attached to an alpha helical peptide bridge type molecular wire as exemplified as follows. In the first step, the bare (unmodified) peptide bridge is suspended in an appropriate aqueous buffer solution including stabilizers or detergents as needed. At room temperature, the cysteine selective clickable bioconjugation reagent is added as a solution in polar, water-miscible solvent. The reaction mixture is then incubated with mild agitation at temperatures ranging from 4 C to 30 C. Once sufficient time has elapsed, the crude reaction mixture is filtered to remove solids, and small molecule impurities are removed via diafiltration. The processed reaction mixture is then purified via preparative reverse phase high pressure liquid chromatography, generally eluting with a gradient of trifluoroacetic acid containing water and acetonitrile. The eluted fractions containing the product of interest are then diluted with water and frozen, followed by lyophilization. The lyophilized product is resuspended in an appropriate buffer solution, ensuring complete neutralization of residual acid. The purified product solution is quantified via gel densitometry and UV-Vis spectrophotometry and purity is assessed by SDS-PAGE and analytical HPLC. In the subsequent step, the purified clickable protein intermediate solution is mixed with a solution of the probe of interest containing a compatible reactive functional group, generally but not limited to an azide. The probe-bridge reaction mixture is then incubated at an appropriate temperature, generally 4° C.-50° C. Once sufficient time has elapsed, the crude reaction mixture is concentrated via diafiltration and purified via Size Exclusion HPLC using an isocratic mobile phase containing appropriate buffer and stabilizers or detergents as needed. The eluted fractions containing the product of interest are then further concentrated and buffer exchanged into the desired final buffer composition via diafiltration. The purified product solution is quantified via gel densitometry and UV-Vis spectrophotometry and purity is assessed by SDS-PAGE and analytical HPLC.

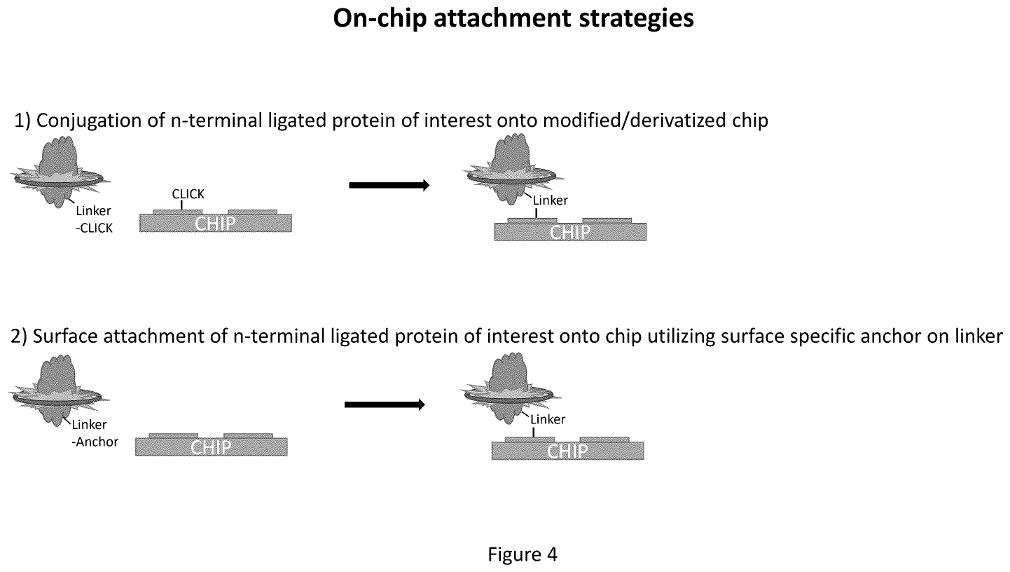

FIG. 4 shows two representative strategies for incorporating probes of interest into the circuit system. First, the probe of interest is tagged at the N-terminus with a polyfunctional linker containing a “click” reactive moiety. Next the clickable probe is introduced to a material that has been previously modified to contain compatible “click” reactive partners, thus ligating the probe into the circuit. Alternatively, the N-terminus tagged probe can be inserted into the system via incorporation of material specific anchors into the polyfunctional linker.

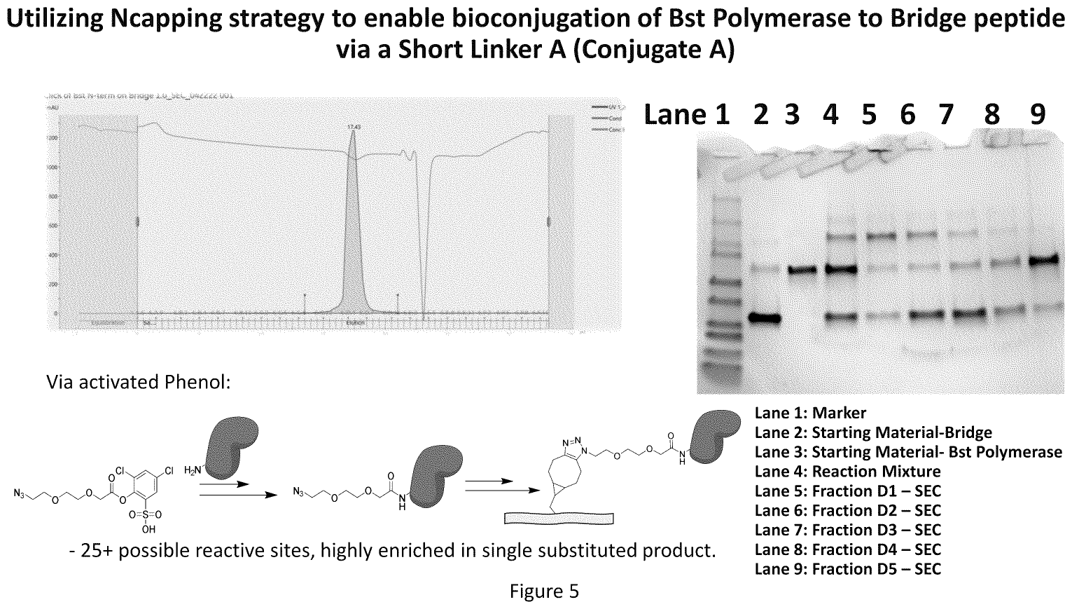

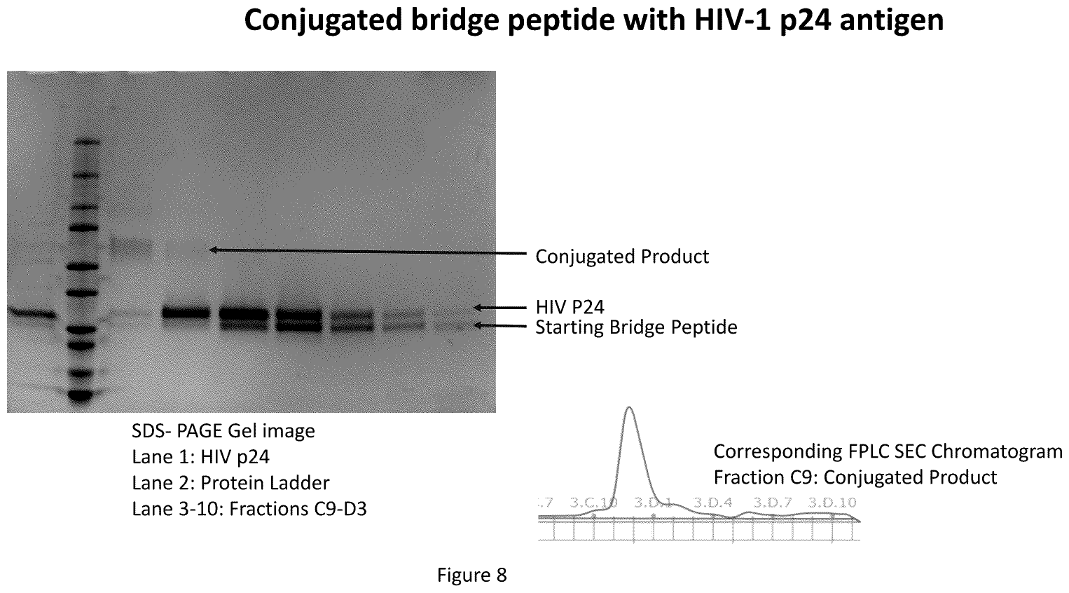

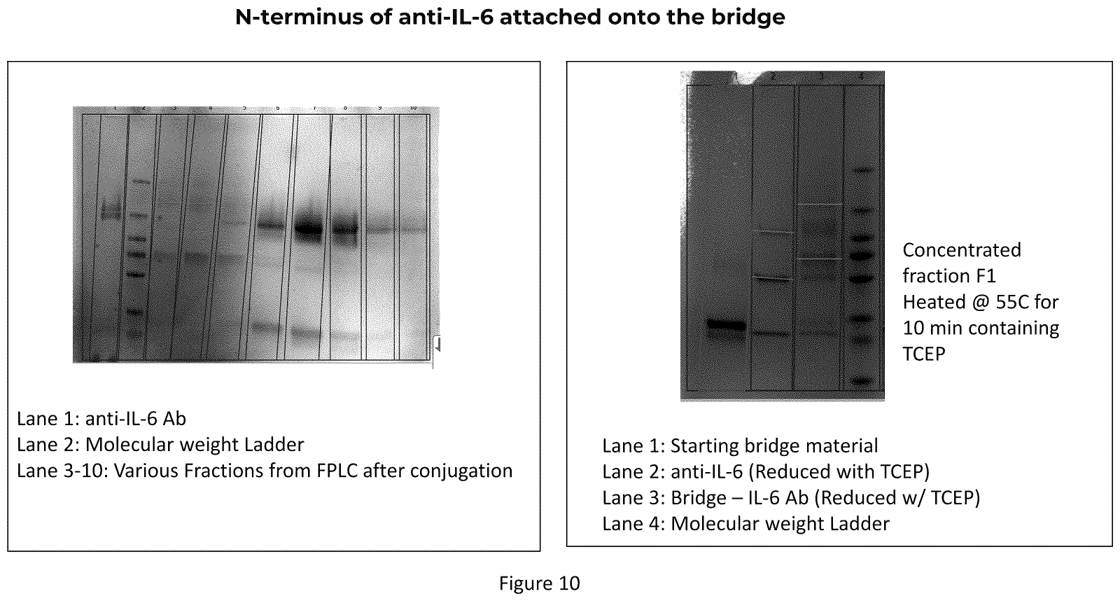

Protein-Bridge Conjugation Examples Along with Appropriate Activity/Binding Assays (FIGS. 5-12):

To show versatility of N-terminal conjugation chemistry, we have demonstrated a variety of model proteins and enzymes, including polymerase, antigen, antibody, and the corresponding relevant biochemical and binding assays.

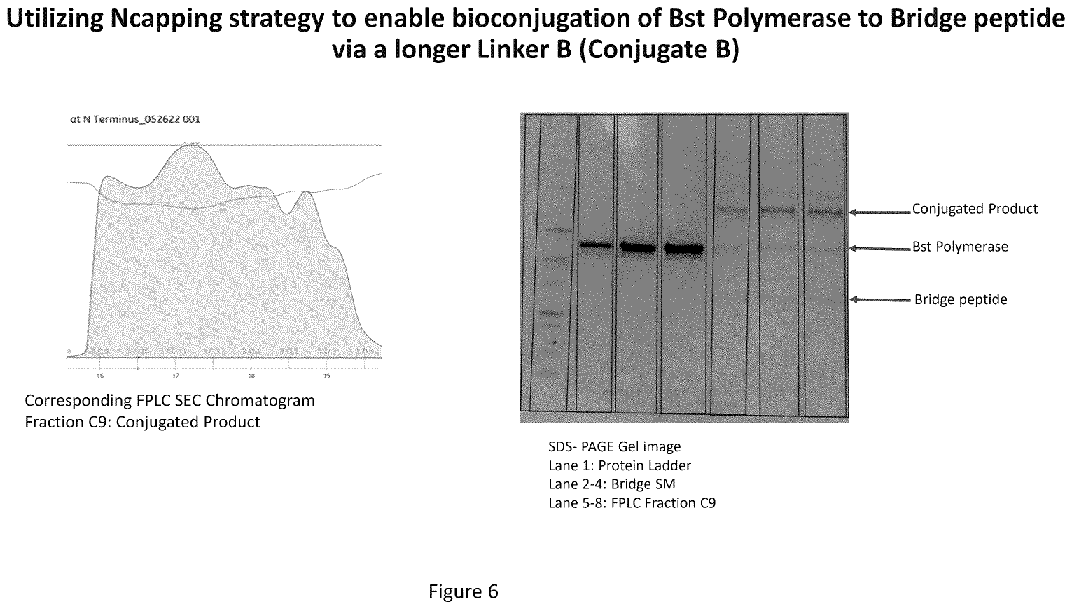

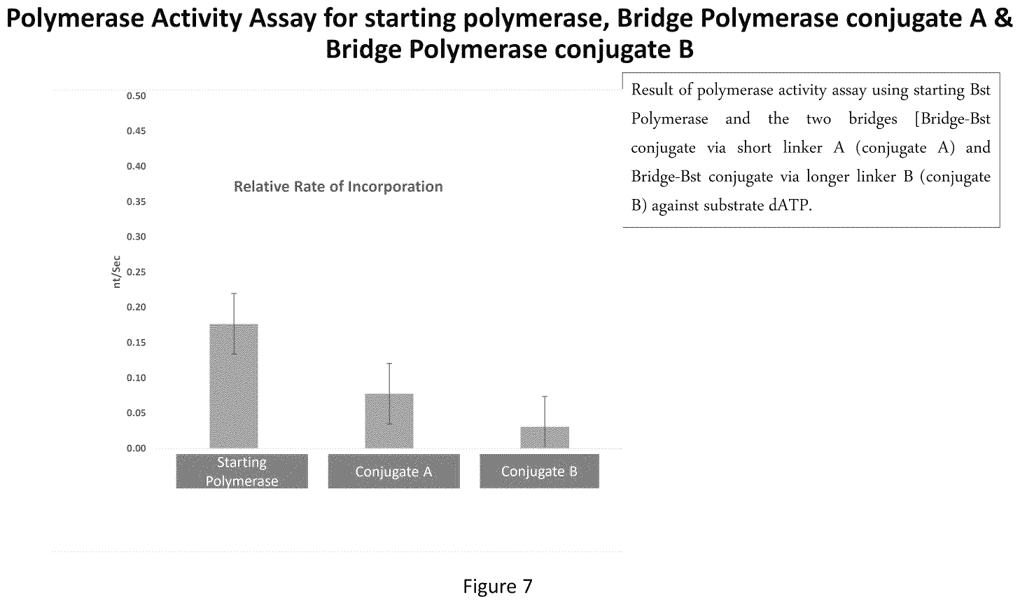

FIG. 5-7 depicts example of conjugation to an exemplary bridge peptide and corresponding activity assay for DNA polymerase. FIG. 5 shows the selective conjugation of Bst polymerase with a modified short linker A to the molecular wire. FIG. 6 shows the conjugation of the same polymerase with another modified longer linker B to the molecular wire. The result of polymerase activity assay highlighting relative rate of incorporation for the two conjugated materials against the starting polymerase is shown in FIG. 7. As evident, the two N-terminally conjugated enzymes retain substantial polymerase activity.

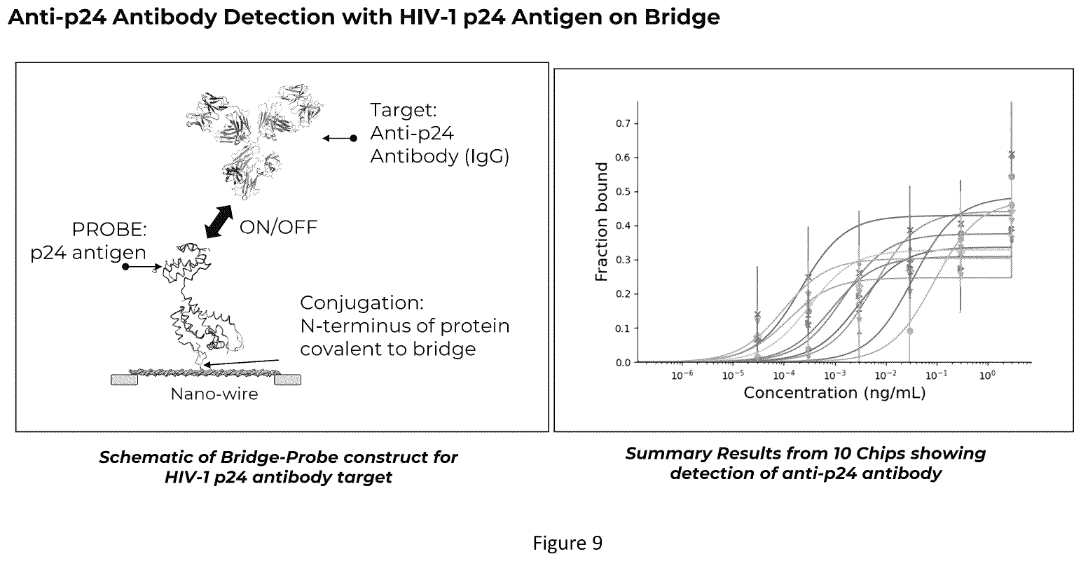

In the scheme shown in FIG. 9, where an antigen is attached to the bridge, we show protein-protein binding. The probe on the peptide bridge is HIV p24 antigen having an affinity for anti-p24 antibodies. In the presence of the target anti-p24 antibodies, the sensor current exhibits pulses corresponding to antigen-antibody binding events. The rate of pulse detection (and the fraction of time in the bound state) increases with higher target molecule concentration. Plotting the fraction of the time bound versus the target concentration produces a typical binding response curve for this antigen-antibody interaction.

In the scheme shown in FIG. 11, where an antibody is attached to the bridge, we show antibody-antigen binding. The probe on the peptide bridge is an anti-Interleukin-6 antibody having affinity for Interleukin-6 antigens. In the presence of the interleukin-6 target antigen, the sensor current exhibits pulses corresponding to antibody-antigen binding events. Plotting the fraction of the time bound versus the target concentration produces a typical binding response curve for this antibody-antigen interaction.

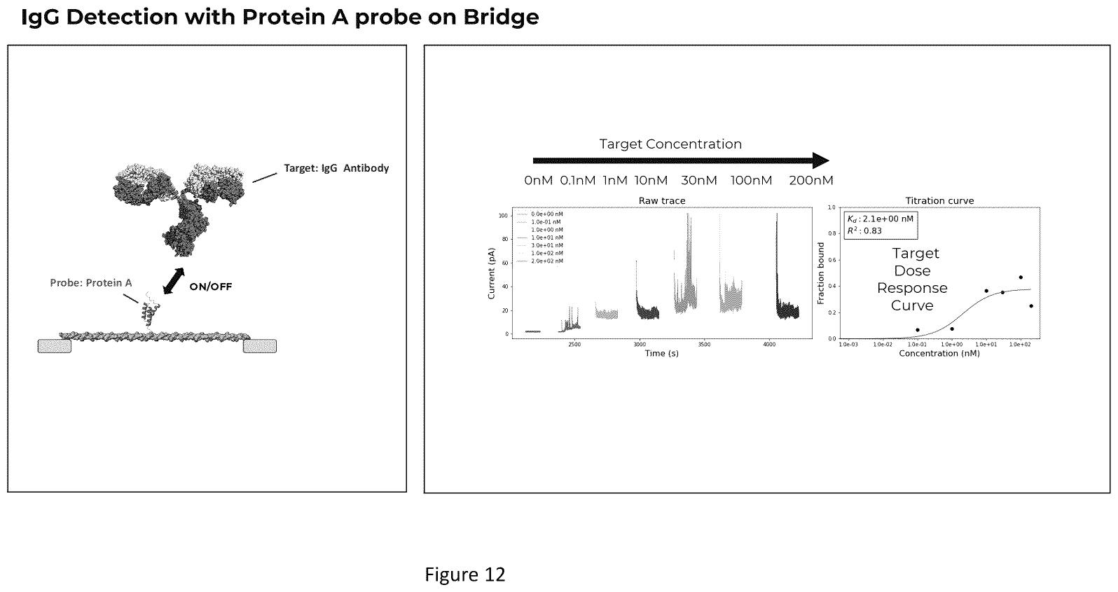

In the scheme shown in FIG. 12, the probe on the peptide bridge is Protein A. Protein A has remarkable ability to bind with the constant (Fc) portion of immunoglobulin molecules from several different species. In the presence of the IgG antibody, the sensor current exhibits pulses corresponding to antigen-antibody binding events. Plotting the fraction of the time bound versus the target concentration produces a typical binding response curve for this antigen-antibody interaction.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A biosensor device comprising:
a current carrying molecular structure comprising a metal contact on the surface of an electrode, wherein the metal contact is coupled to a molecular wire that comprises a conjugation site;
a binding probe molecule conjugated to the molecular wire at the conjugation site, wherein the conjugation of the binding probe molecule to the molecular wire is through a polyfunctional linker molecule covalently linked at one end to the N-terminus of the binding probe molecule and at the other end to the conjugation site in the molecular wire; and
wherein a circuit is formed capable of detecting and/or obtaining detailed information about the binding of the binding probe molecule to a target ligand or binding partner.

2. A biosensor device according to claim 1, wherein the current carrying structure comprises i) a positive electrode having a metal contact on its surface, and ii) a negative electrode having a metal contact on its surface.

3. A biosensor device according to claim 1, wherein the molecular wire is an alpha helical peptide or polynucleotide.

4. A biosensor device according to claim 1, wherein the molecular wire is an alpha helical peptide.

5. A biosensor device according to claim 1, wherein the molecular wire is a non-peptide comprising graphene, Indium oxide thin film ribbons, a carbon nanotube, a single or double stranded polynucleotide, or DNA origami.

6. A biosensor device according to claim 4, wherein the alpha helical peptide has a conjugation site amino acid for coupling to a polyfunctional linker molecule.

7. A biosensor device according to claim 6, the conjugation site amino acid is selected from a Cysteine (Cys, C), Lysine (Lys, K), or Tyrosine (Tyr, T) amino acid residue.

8. A biosensor device according to claim 1, wherein the polyfunctional linker molecule comprises one or more chemically reactive moieties or surface modifying/binding motifs.

9. A biosensor device according to claim 1, wherein the polyfunctional linker molecule comprises a vinyl boronate linker.

10. A biosensor device according to claim 1, wherein the polyfunctional linker molecule comprises an activated phenol linker.

11. A biosensor device according to claim 8, wherein one or more reactive moiety comprises a mixed anhydride comprising a sulfonamide or sulfonic acid that is meta to the phenolic oxygen, and proximal to one or more electron withdrawing groups, including but not limited to halogens, polyfluorinated hydrocarbons, nitriles, carboxylic acids and derivatives, and nitro groups.

12. A biosensor device according to claim 1, wherein the polyfunctional linker molecule comprises a reactive moiety is comprising a E-styrenyl with a para-electron donating moiety, or an E-vinyl boronate derived from an α-nucleophile/electrophile alkyne that is reacted with a linker that contains one or more reactive groups that can undergo selective ‘click’ style chemistries and be utilized as a scaffold for further expansion.

13. A biosensor device according to claim 1, wherein the binding probe molecule comprises a protein, peptide, polypeptide, or protein complex of biological or synthetic origin.

14. A biosensor device according to claim 1, wherein the binding probe molecule comprises a polymerase.

15. A biosensor device according to claim 1, wherein the binding probe molecule comprises a viral antigen.

16. A biosensor device according to claim 1, wherein the binding probe molecule comprises an antibody.

17. A biosensor device according to claim 1, wherein the binding probe molecule is selected from a DNA polymerase, the HIV-1 p24 viral antigen, an anti-IL-6 antibody or binding fragment thereof, or protein A.

18. A molecular wire and binding probe molecule conjugate for use in a biosensor, wherein the molecular wire comprises an alpha helical peptide having a conjugation site, and wherein the binding probe molecule is a protein having a N-terminus, and wherein the conjugation of the binding probe molecule to the alpha helical peptide is through a polyfunctional linker molecule covalently linked at one end to the N-terminus of the binding probe molecule and at the other end to a conjugation site in the alpha helical peptide.

19. A molecular wire and binding probe molecule conjugate according to claim 18, wherein the binding probe molecule comprises a protein selected a DNA polymerase, the HIV-1 p24 viral antigen, an anti-IL-6 antibody or binding fragment thereof, or protein A.

20. A method of making a molecular wire and binding probe conjugate according to claim 18, the method comprising the steps of i) synthesis of a polyfunctional linker molecule comprising one or more chemically reactive handles, ii) select a binding probe molecule of interest, iii) react the binding probe molecule with bifunctional linker reagent under selected conditions to produce a clickable intermediate binding probe molecule suitable for carbon-hetero bond formation reactions or click chemistry, iv) react the clickable intermediate binding probe molecule with a pre-selected molecular wire under appropriate reaction conditions, and v) purify the molecular wire and binding probe conjugate.
